(19) United States
(12) Patent Application Publication
(10) Pub. No.: US 2013/0018124 A1
(43) Pub. Date: Jan. 17, 2013

(54) PROCESS TO MAKE BIODEGRADABLE A SYNTHETIC POLYMER

(71) Applicant: Madrisano Catinari, Civitanova Marche (IT)

(72) Inventor: Madrisano Catinari, Civitanova Marche (IT)

(73) Assignee: QUEENSBROOK LIMITED, Dublin 4 (IE)

(21) Appl. No.: 13/574,404

(22) PCT Filed: Jan. 25, 2011
(86) PCT No.: PCT/IB2011/050310

§ 371 Date: Oct. 2, 2012

(30) Foreign Application Priority Data
Jan. 25, 2010 (IT) AN2010A000002

(51) Int. Cl. C12P 13/00; C12P 7/62; C12N 1/16; C12P 17/16; C12P 5/02; C12P 9/00; C12P 13/02

(52) U.S. Cl. 523/124; 435/128; 435/131; 435/135; 435/129; 435/118; 435/167; 435/255.1

(57) ABSTRACT

A process for making biodegradable a synthetic polymeric material thanks to the addition of one or more yeasts to the synthetic material is disclosed.

The present invention refers to a process for making biodegradable a synthetic polymeric material.

It is well-known that plastic materials, due to their extreme versatility, low cost and mechanical properties, have spread in a remarkable manner, permeating virtually all sectors of our life. It is also known that precisely the chemical and heat resistance of these materials—which property makes them particularly attractive for the industry—also makes the disposal thereof difficult, since enormously long times are needed for their degradation, so that an actual problem of soiling arises, due to plastic material waste.

Various attempts have been made to try and solve this problem, which is becoming increasingly serious.

Initially, attempts have been directed at creating water-soluble plastic materials, so that their release into the sea or their exposure to rain led to their disappearance. However, such materials, in addition to not being usable, precisely because of their solubility, for a number of applications, did solve the problem of soiling, but causing pollution of water streams and of water resources in general.

In a subsequent phase, attempts have been directed at obtaining photo-degradable plastic materials which, when exposed to light, tended to degrade into their monomeric components. However, also this solution often led to greater pollution, since monomers are often toxic agents and in any case the diffusion thereof in the soil and in groundwater tables could not be controlled.

Starch-based plastic materials have been manufactured subsequently, such as for example the so-called MaterBi by Novamont. However, in addition to posing problems of using food crops as raw material (thus subtracting them from their main and vital use), they had a rigidity which made them unsuitable for the main uses.

The use of suitable natural plasticisers, proposed by the present Applicant (PCT/IT2005/000166, Italian patent application No. AN2008A 000024), managed to solve the rigidity problem, making these materials sufficiently resilient to allow their use in the most diverse applications. However, the serious problem of the debatable supply of raw materials remains. Moreover, such plastic materials are remarkably more expensive than common synthetic polymeric materials.

In a subsequent attempt, the Applicant proposed, with Italian patent application No. AN2008A 000013, to functionalise through proteins the majority of synthetic plastic materials, so as to make them biodegradable. However, the results thus obtained are fully unsatisfactory since the products obtained have proved not sufficiently biodegradable.

WO2007129861 describes a foamed polyurethane comprising yeasts, aimed at reducing the generation of volatile organic compounds generated by the foamed material. No mention is made of any biodegradability properties of the material obtained.

U.S. Pat. No. 4,605,622 describes a process for fastening microorganisms to a printed granular item, wherein there are yeasts among the microorganisms. However, the item acts as support for the use as catalyst of the microorganism and does not address the problem of waste disposal.

A similar problem is addressed and solved in EP 0052829, which does not address waste disposal either.

The problem at the basis of the invention is to propose a process which allows to make biodegradable a synthetic polymeric material, which does not require to use directly food raw materials, which implies low manufacturing costs and high performances.

This object is achieved by means of a process for making biodegradable a synthetic polymeric material, characterised in that it comprises the addition of one or more yeasts to the synthetic material.

The present invention refers also to a process for the manufacturing of such polymeric material.

The process according to the present invention provides the mixing of yeasts to a plastic material. Such addition does not affect the mechanical and heat resistance properties of the materials. Preferably, such addition of yeasts is performed before the addition of plasticisers. In some cases, such addition is made to one or more of its monomers, before the polymerisation reaction. Such mixing does not affect normal polymerisation conditions.

All yeast strains may be used for the present invention. In particular, good results have been obtained with the yeast strains Kluyveromyces fragilis and Saccharomyces cerevisiae (beer yeast).

The plastic materials which the present invention can be applied to are all the plastic materials which have functional groups, such as polyurethanes, thermoplastic polyurethanes, PVC, polyethyenterephtalate, polypropylenterephtalate, copolymer ethylen and vinyl-acetate (EVA), nylon, rayon. Moreover, the present invention can be applied also to materials which do not have functional groups, such as polyethylene and polypropylene.

The addition, for example, in the case of polyurethane, occurs even before polymerisation in the polyol, while in the case of PVC and, in general, of extruded polymers, it is added to the already polymerised raw product, before extrusion, preferably by dry grinding.

Yeasts are to be added, preferably live, in an amount ranging between 0.3 and 30% of the overall weight of the monomer, preferably between 2 and 7% of the overall weight of the monomer, even more preferably 5% of the overall weight of the monomer.

The polymeric material thus obtained exhibits high-biodegradability properties. Without wanting to be tied to theory, it is believed that yeasts structurally transform the final polymer, making it attackable by the bacteria contained in the soil and/or in the middle of the other waste, so that biodegradation occurs in a short time and at a very high rate. Yeasts are living organisms, but not essential per se to human nutrition, so that their use does not cause problems of food resource shortages. Usable yeasts do not have toxic properties nor do they carry diseases, so the plastic material according to the present invention can be used also in the food and/or pharmaceutical sectors. Moreover, the material according to the present invention can be used for a number of other purposes, since it can be plasticised at will, originating a final product of the desired rigidity.

Yeasts, in particular those of the strains Saccharomyces cerevisiae and Kluyveromyces fragilis, are easily available, inexpensive and very easy to handle, not posing health and/or environmental hazards. The plastic materials which are obtained may be manufactured at the low costs which are common in connection with synthetic plastic materials and are hence less expensive than the ones obtained, for example, from corn starch, despite having the same biodegradability.

The present invention extends also to a manufacturing process of a biodegradable polymeric material, comprising the standard processing stages specific of the particular plastic material, furthermore comprising the addition to the monomer, before polymerisation, of one or more yeasts.

The present invention refers also to a polymerisation kit, comprising one or more yeasts. Said kit advantageously contains an instructions manual reporting concentrations. Even more advantageously, said kit contains an amount of yeasts dosed so as to make biodegradable an amount of plastic material reported on the package.

However, it is understood that the invention must not be considered limited to the particular arrangement illustrated above, which makes up only an exemplifying embodiment thereof, but that a number of variants are possible, all within the reach of a person skilled in the field, without departing from the scope of this invention, as defined by the following claims.

### EXAMPLE

Equivalent amounts (50%-50% in moles) of a polyol and of an isocianate were prepared for the manufacturing of a polyurethane. To the polyol 5% of its overall weight of Saccharomyces cerevisiae was added. The two components were then mixed, caused to react and polymerise in the usual reaction conditions. A polyurethane was obtained which was moulded in a sheet. The sheet was crumbled and subjected to the conditions provided by technical rules. After 54 days it proved to be biodegradable in composting, reaching an average biodegradability value in excess of 90% as required by rule UNI EN 13432:2000 (par. A.2.2.2).

1. Process for making biodegradable a synthetic polymeric material, characterised in that it comprises the addition to the synthetic material of one of more yeasts.

2. Process as claimed in claim 1, characterised in that said one or more yeasts are added before the addition of plasticiser to the synthetic material.

3. Process as claimed in claim 2, characterised in that said yeasts are added to one or more of the monomers, before the polymerisation reaction.

4. Process as claimed in claim 1, characterised in that said synthetic polymeric material is chosen in the group consisting of polyurethanes, PVC, polyethyleneterephtalate, polypropyleneterephtalate, of co-polymer ethylene and vinyl acetate (EVA), nylon, rayon, polyethylene, polypropylene.

5. Process as claimed in claim 4, characterised in that said polymeric material is polyurethane and in that the yeast is added to the polyol before polymerisation.

6. Process as claimed in claim 1, characterised in that said yeasts are Kluyveromices fragilis and/or Saccharomyces cerevisiae.

7. Kit for the implementation of a process according to claim 1, characterised in that it contains one or more yeasts, in a dosed amount.

8. A biodegradable synthetic polymeric material comprising yeasts.

9. The biodegradable synthetic polymeric material as claimed in claim 8, characterised in that said yeasts are Kluyveromices fragilis and/or Saccharomices cerevisiae.
